(19) United States
(12) Patent Application Publication
(10) Pub. No.: US 2008/0064624 A1
(43) Pub. Date: Mar. 13, 2008

(54) Organoleptic Compounds and Their Use in Perfume Compositions

(76) Inventors: Anubhav P.S. Narula, Hazlet, NJ (US); Edward Mark Arruda, Cliffwood, NJ (US)

Correspondence Address: INTERNATIONAL FLAVORS & FRAGRANCES INC., 521 WEST 57TH ST, NEW YORK, NY, 10019, US

(21) Appl. No.: 11/530,645

(22) Filed: Sep. 11, 2006

(51) Int. Cl. A61K 8/18

(52) U.S. Cl. 512/20

(57) ABSTRACT

The present invention is directed to the novel cyclohexyl, cyclopentyl and acyclic ester compounds of the general formula:

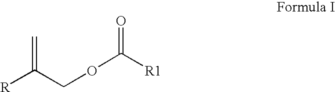

wherein R and R¹ independently represent a hydrogen or a straight, branched or cyclic hydrocarbon consisting of less than 15, preferably less than 10, most preferably less than 4 carbon atoms and contain one or more double bonds.

### FIELD OF THE INVENTION

The present invention relates to new chemical entities and the incorporation and use of the new chemical entities as fragrance materials.

### BACKGROUND OF THE INVENTION

There is an ongoing need in the fragrance industry to provide new chemicals to give perfumers and other persons ability to create new fragrances for perfumes, colognes and personal care products. Those with skill in the art appreciate how differences in the chemical structure of the molecule can result in significant differences in the odor, notes and characteristics of a molecule. These variations and the ongoing need to discover and use the new chemicals in the development of new fragrances allows perfumers to apply the new compounds in creating new fragrances.

### SUMMARY OF THE INVENTION

The present invention provides novel chemicals, and the use of the chemicals to enhance the fragrance of perfumes, toilet waters, colognes, personal products and the like. In addition, the present invention is directed to the use of the novel chemicals to enhance fragrance in perfumes, toilet waters, colognes, personal products and the like.

In one embodiment, the present invention is directed to the novel compounds represented by the general Formula I set forth below:

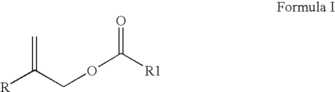

wherein R and R¹ independently represent a hydrogen or a straight, branched or cyclic hydrocarbon consisting of less than 15, preferably less than 10, most preferably less than 4 carbon atoms and may contain one or more double bonds.

Another embodiment of the invention is a method for enhancing a perfume composition by incorporating an olfactory acceptable amount of the compounds provided above.

These and other embodiments of the present invention will be apparent by reading the following specification.

### DETAILED DESCRIPTION OF THE INVENTION

In Formulae I above R and R¹ independently represent a hydrogen or a straight, branched or cyclic hydrocarbon consisting of less than 15, preferably less than 10, most preferably less than 4 carbon atoms and may contain one or more double bonds. Suitable straight hydrocarbons include alkanes such as but not limited to ethyl, propyl, butyl, pentyl, hexyl and the like. Suitable branched hydrocarbons include alkanes such as but not limited to isopropyl, sec-butyl, tert-butyl, 2-ethyl-propyl and the like. Suitable alkenes containing double bonds include ethene, propene, 1-butene, 2-butene, penta-1-3-deine, hepta-1,3,5-triene and the like. Cyclic hydrocarbons include cyclopropyl, cyclobutyl, cyclohexyl, phenyl and the like.

In the most preferred embodiment of the invention, the novel compound of the present invention is represented by the following structure:

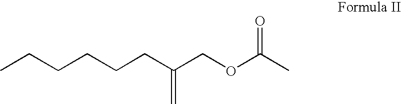

Those with the skill in the art will appreciate that the compound of Formula II is 2-Methylene-1-Octanol Acetate.

Formula II has the following fragrance notes:

| Fresh | Dry |
|---|---|
| Aldehydic, clonal, citrus, ozone, fatty, fresh, floral, fruity, | Orange, |
| slight mushroom, citrusy nitrile slightly rosalva, rose alcohol | mandarin, |
| | waxy, |

The table below lists additional compounds derived from Formula II that are described in the present invention:

| Structure | Name |
|---|---|
| 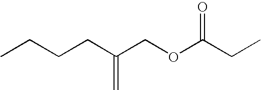 | Propanoic acid 2-methylene-hexyl ester |
| 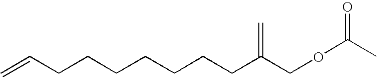 | 10-undecen-1-ol, 2-Methylene-, Acetate |
| 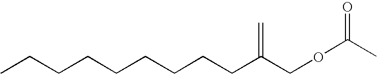 | Acetic acid 2-methylene-undecyl ester |
| 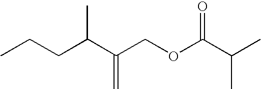 | Isobutyric acid 3-methyl-2-methylene-hexyl ester |
| 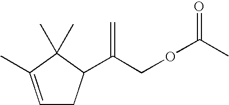 | 3-cyclopentene-1-ethanol,2,2,3-trimethyl-beta-methylene acetate |
| 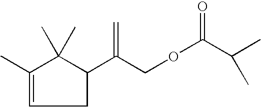 | Isobutyric acid 2-(2,2,3-trimethyl-cyclopent-3-enyl)-allyl ester |
| 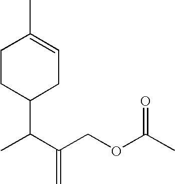 | Acetic acid 3-(4-methyl-cyclohex-3-enyl)-2-methylene-butyl ester |
| 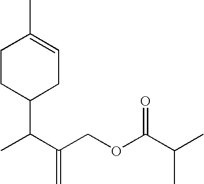 | Isobutyric acid 3-(4-methyl-cyclohex-3-enyl)-2-methylene-butyl ester |
| 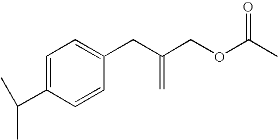 | Acetic acid 2-(4-isopropyl-benzyl)-allyl ester |
| 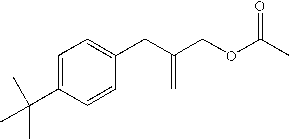 | Acetic acid 2-(4-tert-butyl-benzyl)-allyl ester |
| 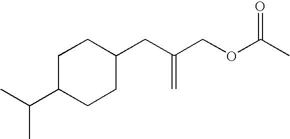 | Acetic acid 2-(4-isopropyl-cyclohexylmethyl)-allylester |
| 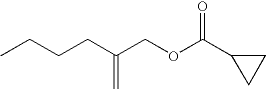 | Cyclopropanecarboxylic acid 2-methylene-hexylester |
| 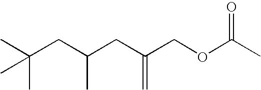 | Acetic acid 4,6,6-trimethyl-2-methylene-heptyl ester |
| 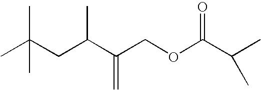 | 1-Hexanol,3,5,5-trimethyl-2-methylene; isobutyrate |
| 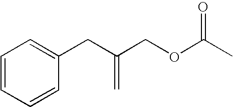 | 2-propen-1-ol, 2-benzyl acetate |
| 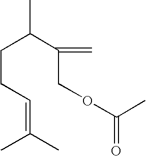 | 6-octen-1-ol,3,7-dimethyl-2-methylene acetate |
| 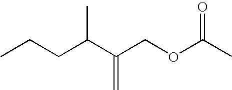 | 1-hexanol, 3-methylene-, acetate |
| 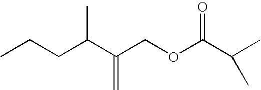 | Propanoic acid, 2-methyl-(4Z)-4-octenyl ester |
| 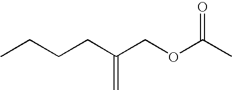 | 1-Hexanol, 5,5-deimethyl-3-methylene-, formate |

The compounds of the present invention may be prepared from the corresponding compounds via an Acetylation reaction using the following sequence:

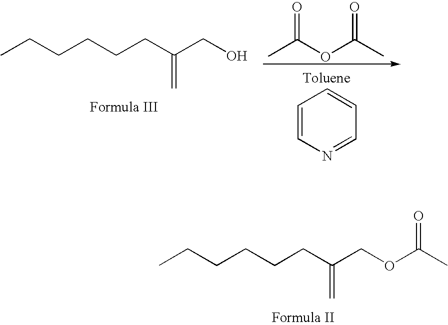

The starting materials, for the above reaction, are Formula III which is 99% pure 2-methylene-1-octanol, toluene and pyridine. The starting material is commercially available from Aldrich Chemical Company.

Those with skill in the art will recognize that some of the compounds of the present invention have a number of chiral centers, thereby providing numerous isomers of the claimed compounds. It is intended herein that the compounds described herein include isomeric mixtures of such compounds, as well as those isomers that may be separated using techniques known to those having skill in the art. Suitable techniques include chromatography such as HPLC, and particularly gel chromatography and solid phase microextraction (“SPME”).

The use of the compounds of the present invention is widely applicable in current perfumery products, including the preparation of perfumes and colognes, the perfuming of personal care products such as soaps, shower gels, and hair care products as well as air fresheners and cosmetic preparations. The present invention can also be used to perfume cleaning agents, such as, but not limited to detergents, dishwashing materials, scrubbing compositions, window cleaners and the like.

In these preparations, the compounds of the present invention can be used alone or in combination with other perfuming compositions, solvents, adjuvants and the like. The nature and variety of the other ingredients that can also be employed are known to those with skill in the art.

Many types of fragrances can be employed in the present invention, the only limitation being the compatibility with the other components being employed. Suitable fragrances include but are not limited to fruits such as almond, apple, cherry, grape, pear, pineapple, orange, strawberry, raspberry; musk, flower scents such as lavender-like, rose-like, iris-like, carnation-like. Other pleasant scents include herbal and woodland scents derived from pine, spruce and other forest smells. Fragrances may also be derived from various oils, such as essential oils, or from plant materials such as peppermint, spearmint and the like.

A list of suitable fragrances is provided in U.S. Pat. No. 4,534,891, the contents of which are incorporated by reference as if set forth in its entirety. Another source of suitable fragrances is found in Perfumes, Cosmetics and Soaps, Second Edition, edited by W. A. Poucher, 1959. Among the fragrances provided in this treatise are acacia, cassie, chypre, cyclamen, fern, gardenia, hawthorn, heliotrope, honeysuckle, hyacinth, jasmine, lilac, lily, magnolia, mimosa, narcissus, freshly-cut hay, orange blossom, orchid, reseda, sweet pea, trefle, tuberose, vanilla, violet, wallflower, and the like.

Olfactory effective amount is understood to mean the amount of compound in perfume compositions the individual component will contribute to its particular olfactory characteristics, but the olfactory effect of the perfume composition will be the sum of the effects of each of the perfumes or fragrance ingredients. Thus the compounds of the invention can be used to alter the aroma characteristics of the perfume composition, or by modifying the olfactory reaction contributed by another ingredient in the composition. The amount will vary depending on many factors including other ingredients, their relative amounts and the effect that is desired.

The level of compound of the invention employed in the perfumed article varies from about 0.005 to about 10 weight percent, preferably from about 0.5 to about 8 and most preferably from about 1 to about 7 weight percent. In addition to the compounds other agents can be used in conjunction with the fragrance. Well known materials such as surfactants, emulsifiers, polymers to encapsulate the fragrance can also be employed without departing from the scope of the present invention.

Another method of reporting the level of the compounds of the invention in the perfumed composition, i.e., the compounds as a weight percentage of the materials added to impart the desired fragrance. The compounds of the invention can range widely from 0.005 to about 70 weight percent of the perfumed composition, preferably from about 0.1 to about 50 and most preferably from about 0.2 to about 25 weight percent. Those with skill in the art will be able to employ the desired level of the compounds of the invention to provide the desired fragrance and intensity.

The following are provided as specific embodiments of the present invention. Other modifications of this invention will be readily apparent to those skilled in the art. Such modifications are understood to be within the scope of this invention. As used herein all percentages are weight percent unless otherwise noted, ppm is understood to stand for parts per million, g is understood to be grams and Ac₂O is understood to mean acetic anhydride. The odor descriptions of the following fragrance compounds are evaluated both dry and fresh and in Tables 1-7 on an intensity scale of 1-3. IFF as used in the examples is understood to mean International Flavors & Fragrances Inc., New York, N.Y., USA.

### EXAMPLE A

### Preparation of 2-methylene-1-octanol acetate

To a dry 2 L multi-neck round bottom flask fitted with an air stirrer, nitrogen inlet condenser and an addition funnel 380 g of 99% pure 2-methylene-1-octanol, 21.2 g of pyridine and 600ml of toluene. 241 g of acetic anhydride (Ac₂O) was added to the mixture dropwise over 2 hours. A sample was taken every 30 minutes during the addition of the acetic anhydride. The mixture was aged for 2 hours. 600 ml of water was slowly added to the mixture. The mixture was cooled, allowed to settle and the layers were allowed to separate. The mixture was neutralized with 5% NaHCO₃.

The NMR of the 2-methylene-1-octanol acetate is as follows: 00.9 ppm (m, 3H); 1.3 ppm (s, 6H); 1.4-1.5 ppm (m, 2H); 2.0 ppm (m, 2H); 2.1 ppm (s, 3H); 4.5 ppm (s, 2H); 4.9 ppm (s, 1H); 5 ppm (s, 1H).

### Odor Description:

TABLE I

| DRY | INTENSITY | FRESH |
|---|---|---|
| | 3 | |
| Orange, mandarin, waxy | 2 | Mandarin, aldehydic and fruity |
| | 1 | |

### EXAMPLE B

### Preparation of 1-hexanol 3,5,5-trimethyl-2-methylene, isobutyrate

To a dry 2 L multi-neck round bottom flask fitted with an air stirrer, nitrogen inlet condenser and an addition funnel 468 g of 99% pure 2-methylene-3,5,5-trimethyl-1-hexanol, 23 g of pyridine and 500 ml of toluene. 237 g of acetic anhydride was added to the mixture dropwise over 2 hours. A sample was taken every 30 minutes during the addition of the acetic anhydride. The mixture was aged for 2 hours. 500 ml of water was slowly added to the mixture. The mixture was cooled, allowed to settle and the layers were allowed to separate.

The NMR of the 1-hexanol 3,5,5-trimethyl-2-methylene, isobutyrate is as follows: 0.9 ppm (s, 9H); 1.1 ppm (s, 3H); 1.2 ppm (s, 7H); 1.5 ppm (m, 1H); 2.3 ppm (s, 1H); 2.6 ppm (s, 1H); 4.5 ppm (s, 2H); 5 ppm (s, 2H).

### Odor Description:

TABLE 2

| | | |
|---|---|---|
| DRY | INTENSITY | FRESH |

| | |
|---|---|
| 3 | |
| 2 | Fruity, floral and hay-like |
| 1 | |

### EXAMPLE C

### Preparation of 10-undecen-1-ol, 2-methylene-, acetate

To a dry 2 L multi-neck round bottom flask fitted with an air stirrer, nitrogen inlet condenser and an addition funnel 668 g of 99% pure 2-methylene-10-undecen-1-ol, 29 g of pyridine and 1000 ml of toluene. 237 g of Ac₂O was added to the mixture dropwise over 1 hour. At maximum conversion 100 ml of water was added to decompose Ac₂O. The mixture was cooled, allowed to settle and the layers were allowed to separate then washed with organic two times with 500 ml 20% NaHCO₃ solution.

The NMR of the 10-undecen-1-ol, 2-methylene-, acetate. is as follows: 1.2 ppm (s, 6H); 1.4 ppm (s, 2H); 1.5-1.6 ppm (m, 2H); 2.0 ppm (m, 7H); 4.5 ppm (s, 2H); 4.9 ppm (s, 1H); 5 ppm (m, 4H).

### Odor Description:

TABLE 3

| | | |
|---|---|---|
| DRY | INTENSITY | FRESH |

| | |
|---|---|
| 3 | |
| 2 | Fruity, floral and muguet |
| 1 | |

### EXAMPLE D

### Preparation of Alcohol to be Used in Examples D-G

The Mannich products used as the starting materials may also be prepared as described in Petri M. Pihko et al., Mild Organo Catalytic Alpha-Methylenation of Aldehydes, Journal of Organic Chem. 2006, 71,. 2538-2541.

The starting alcohols for Examples D-G may be prepared from the corresponding aldehydes via a Mannich reaction and followed by reduction as delineated in the following generic sequence as described below:

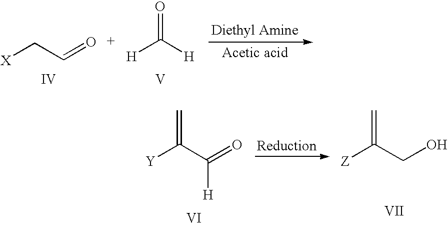

### Wherein IV represents the corresponding aldehyde and X, Y and Z represent the corresponding variable chain, V represents formaldehyde, VI represents an aldehyde and VII represents the corresponding alcohol.

### EXAMPLE E

### Preparation of 3-cyclopentene-1-ethanol, 2,2,3 -trimethyl-beta-methylene acetate

To a dry 2 L multi-neck round bottom flask fitted with an air stirrer, nitrogen inlet condenser and an addition funnel 359 g of acetic anhydride was added, stirred and heated to reflux at 134° C. then 454 g of α methylene compholenic alcohol (VII in the generic sequence of Example D) was added. The sample was heated to 150° C. to remove excess Ac₂O and then the sample was washed over with H2O, 5% sodium bicarbonate and then washed over with brine and yielded 560 grams of crude.

The NMR of the 3-cyclopentene-1-ethanol, 2,2,3-trimethyl-beta-methylene acetate is as follows: 0.8 ppm (s, 3H); 1.08 ppm (s, 3H); 1.6 ppm (s, 3H); 2.09 ppm (s, 3H); 2.23-2.38 ppm (m, 2H); 2.6 ppm (t, 1H); 4.57 ppm (s, 2H); 5.04 ppm (s, 1H); 5.17 ppm (s,1H); 5.29 ppm (s, 1H).

### Odor Description:

TABLE 4

| | | |
|---|---|---|
| DRY | INTENSITY | FRESH |

| | |
|---|---|
| 3 | |
| 2 | Floral, Minty, Muguet, Orivone, |
| | Rose, Vertenex, |
| 1 | Fruity |

### EXAMPLE F

### Preparation of 3-cyclohexene-1-propanol, gamma,4-diemthyl-beta-methylene acetate

To a dry 2 L multi-neck round bottom flask fitted with an air stirrer, nitrogen inlet condenser and an addition funnel 359 g of acetic anhydride was added, stirred and heated to reflux at 134° C. then 360 g of α methylene limonene oxo alcohol (VII in the generic sequence of Example D) was added and then sampled at 138° C. The sample was heated to 150° C. to remove excess Ac₂O and then the sample was washed over with H2O, 5% bicarbonate and then washed over with brine and yielded 266 grams of crude.

The NMR of the 3-Cyclohexene-1-Propanol, Gamma,4-Dimethyl-Beta-methylene Acetate is as follows: 1.01-1.13 ppm (d of d, 3H); 1.4-1.9 ppm (m, 8H); 1.6 ppm (s, 3H); 1.99 ppm (s, 3H); 4.43 ppm (s, 2H); 4.85 ppm (m, 1H); 4.98 ppm (m, 1H); 5.26 ppm (m, 1H).

### Odor Description:

TABLE 5

| DRY | INTENSITY | FRESH |
|---|---|---|
| Lavadaceous, musty, natural | 3 | Floral, mushroomy, musty, |
| mushroomy note | | violet |
| | 2 | green |
| | 1 | |

### EXAMPLE G

### Preparation of 2-Propen-1-ol, 2-Benzyl, Acetate

To a dry 2 L multi-neck round bottom flask fitted with an air stirrer, nitrogen inlet condenser and an addition funnel 359 g of acetic anhydride was added, stirred and heated to reflux at 134° C. then 296 g of 2-propen-1-ol, 2benzyl (VII in the generic sequence of Example D) was added and then sampled at 138° C. The sample was heated to 150° C. to remove excess Ac₂O and then the sample was washed over with H2O, 5% bicarbonate and then washed over with brine and yielded 266 grams of crude.

The NMR of the 2-Propen-1-ol, 2-Benzyl, Acetate is as follows: 1.91 ppm (s, 3H); 3.33 ppm (s, 2H); 4.39 ppm (s, 2H); 4.85 ppm (d, 1H); 5.03 ppm (d, 1H); 7.00-7.29 ppm (m, 5H).

### Odor Description:

TABLE 6

| DRY | INTENSITY | FRESH |
|---|---|---|
| Citrus, lemony, nitrile | 3 | Citrus, lemony, nitrile |
| Citral, lemongrass | 2 | |
| | 1 | |

### EXAMPLE H

### Preparation of 6-Octen-1-ol, 3,7-Dimethyl-2-Methylene Acetate

To a dry 2 L multi-neck round bottom flask fitted with an air stirrer, nitrogen inlet condenser and an addition funnel 359 g of acetic anhydride was added, stirred and heated to reflux at 134° C. then 336 g of, 2-methylene-3,7-dimethyl-6-octen-1-ol (VII in the generic sequence of Example D) was added and then sampled at 138° C. The sample was heated to 150° C. to remove excess Ac₂O and then the sample was washed over with H2O, 5% sodium bicarbonate and then washed over with brine and yielded 266 grams of crude.

The NMR of the 6-Octen-1-ol, 3,7-Dimethyl-2-Methylene Acetate is as follows: 1.04 ppm (d, 3H); 1.2-1.52 ppm (m, 2H); 1.6 ppm (s, 3H); 1.66 ppm (s, 3H); 1.7-2.4 ppm (m, 3H); 2.03 ppm (s, 3H); 4.5 ppm (s, 2H); 4.95 ppm (2s,2H); 5 ppm (m, 1H).

### Odor Description:

TABLE 7

| DRY | INTENSITY | FRESH |
|---|---|---|
| Fatty, slightly citrus, lemony, | 3 | |
| citronellyl acetate | | |
| | 2 | |
| | 1 | Acidic, fruity, citrus |

### EXAMPLE I

### Demonstration Fragrance Formula for a Shower GEL with 2-Methylene-1-Octanol Acetate

| | |
|---|---|
| Agrea | 5.00 |
| Ald C-10 Firm | 5.00 |
| Ald C-12 Lauric “PFG” | 100.00 |
| Citral Refined HLR | 8.00 |
| Dihydro Myrcenol | 60.00 |
| Dodecylene Ald, Alpha Beta BHA | 10.00 |
| Hexenal, Trans 2, 1% DPG | 100.00 |
| Hexenol, B, Gamma Extra | 12.00 |
| Lemon Terpenes Dist “PFG” | 60.00 |
| Lime Dist WI “PFG” | 6.00 |
| Lime Oil Persian MPF “PFG” BHT | 100.00 |
| Mandaril (Elinics) | 50.00 |
| Meth Nonyl Acetald | 12.00 |
| Orange Oil FLA CP “PFG” | 37.00 |
| Terpinolene P UB BHT | 45.00 |
| 2-Methylene-1-Octanol Acetate | 28.00 |
| Total weight | 1000.00 |

What is claimed is:

1. A compound of formula 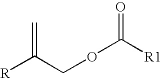
wherein R and R¹ independently represent a hydrogen or a straight, branched or cyclic hydrocarbon consisting of less than 15 carbon atoms and may contain one or more double bonds.

2. A compound of claim 1, wherein R and R¹ independently represent a hydrogen, or a straight, branched or cyclic hydrocarbon consisting of less than 10 carbon atoms.

3. A compound of claim 1, wherein R and R¹ independently represent a hydrogen, or a straight, branched or cyclic hydrocarbon consisting of less than 4 carbon atoms.

4. A method of modifying a fragrance formulation through the addition of an olfactory acceptable amount of the compound of claim 1.

5. The method of claim 4 wherein the fragrance is incorporated into a product selected from perfumes, colognes, toilet waters, cosmetic products, personal care products, fabric care products, cleaning products and air fresheners.

6. The method of claim 5 wherein the cleaning product is selected from the group consisting of detergents, dishwashing compositions, scrubbing compounds and window cleaners.

7. The method of claim 4, wherein the amount incorporated into a fragrance is from about 0.005 to about 10 weight percent.

8. The method of claim 4, wherein the amount incorporated into a fragrance is from about 0.5 to about 8 weight percent.

9. The method of claim 4, wherein the amount of incorporated into a fragrance is from about 1 to about 7 weight percent.

10. A fragrance formulation containing an olfactory effective amount of the compound of claim 1.

11. A fragrance product containing a compound of claim 1.

12. A compound 2-Methylene-1-Octanol Acetate.

13. A fragrance formulation containing an olfactory effective amount of the compound of claim 12.

14. A fragrance product containing a compound of claim 12.
